SEQUENCE LISTING (4 sequences)

SEQ ID NO: 1 - protein, 419 aa, SARS-CoV-2 virus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 222 aa, SARS-CoV-2 virus
MAGSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, SARS-CoV-2 virus
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 1273 aa, SARS-CoV-2 virus
MFVFLVLLPLVSSQCVNLTTRTQLPPAYTNSFTRGVYYPDKVFRSSVLYSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2022/0187279 A1
(43) Pub. Date: Jun. 16, 2022

(54) RAPID T-CELL ASSAY FOR SARS-COV-2

(71) Applicant: ImmunityBio, Inc., Culver City, CA (US)

(72) Inventors: Peter Allan Sieling, Culver City, CA (US); Kayvan Niazi, Culver City, CA (US); Helty Adisetiyo, Culver City, CA (US); Philip T. Liu, Culver City, CA (US)

(21) Appl. No.: 17/544,586

(22) Filed: Dec. 7, 2021

Related U.S. Application Data
(60) Provisional application No. 63/154,314, filed on Feb. 26, 2021.
(60) Provisional application No. 63/124,979, filed on Dec. 14, 2020.

(51) Int. Cl. G01N 33/50

(52) U.S. Cl. CPC G01N 33/505; G01N 2800/26

(57) ABSTRACT

Methods for detecting the presence of sever acute respiratory coronavirus 2 (SARS-CoV-2) are provided. The methods comprise incubating a sample comprising T-cells from an individual with one or more SARS-CoV-2 antigens, each antigen having a T-cell epitope, and detecting a change in state of T-cells in the sample. The change in state may be detected by determining the presence or level of a secreted, immune response indicator, such as interferon-gamma (IFN-γ).

### CROSS-REFERENCE TO RELATED APPLICATIONS

This application claims the benefit of priority under 35 U.S.C. § 119(e) to U.S. Provisional Patent Application No. 63/124,979, filed Dec. 14, 2020 and to U.S. Provisional Patent Application No. 63/154,314, filed Feb. 26, 2021. The entire disclosure of each of U.S. Provisional Patent Application Nos. 63/124,979 and 63/154,314 are incorporated herein by reference.

### REFERENCE TO A SEQUENCE LISTING

This application contains references to amino acid sequences and/or nucleic acid sequences which have been submitted concurrently herewith as the Sequence Listing submitted electronically as a text file named “8774-17_Sequence_Listing_ST25.txt,” file size in bytes of 18,000 bytes, created on Dec. 7, 2021. The information contained in the text file is incorporated herein by reference in its entirety pursuant to 37 C.F.R. § 1.52(e)(5).

### FIELD

The present disclosure generally relates to SARS-CoV-2 diagnostics. In particular, the disclosure relates to detecting SARS-CoV-2 by determining the presence of anti-SARS-CoV-2 T-cells.

### BACKGROUND

In December of 2019, several cases of pneumonia having unknown etiology were reported in China. By early January, these cases, and many others, were discovered to be caused by a novel coronavirus later named severe acute respiratory syndrome coronavirus 2 (SARS-CoV-2). The respiratory disease caused by SARS-CoV-2 was named Coronavirus Disease 2019, or COVID-19. Despite attempts to contain the disease, exponential growth of infected individuals led the World Health Organization to declare COVID-19 a global pandemic in March of 2020.

Rapid spread of SARS-CoV-2 resulted in a high number of deaths throughout the world. The significance of asymptomatic and pre-symptomatic transmission resulted in rapid development of various diagnostic assays, with the goal of mass screening of the population and isolation of individuals known to be infected. These assays broadly fall into the categories of virus detection and antibody detection. Assays for virus detection include those that detect SARS-CoV-2 nucleic acid (i.e., RNA) or viral antigens. Antibody detection assays detect antibodies to SARS-CoV-2, usually antibodies directed to proteins on the surface of the virus. Current tests have proved to have large problems with accuracy, due, perhaps, to problems with the level of vial antigens, anti-virus antibodies, the stability of viral nucleic acid molecules, or the multi-step nature of the tests. Such issues have resulted in a potentially large number of false negative and positive test results.

Thus, a need exists for a SARS-CoV-2 diagnostic test that is easy to use, and that reliably identifies individuals infected with the virus. The present disclosure addresses this need and provides additional benefits as well.

### SUMMARY

Novel methods and compositions are provided that allow for rapid and easy detection of SARS-CoV-2.

One embodiment relates to a method of detecting SARS-CoV-2 in an individual, the method comprising: incubating a sample in a container under conditions sufficient for specific recognition by T-cells, wherein the sample comprises T-cells from the individual, wherein the container comprises one or more SARS-CoV-2 antigens, and wherein the one or more SARS-CoV-2 antigens are deposited on the interior surface of the container and wherein the SARS-CoV-2 antigens comprise T-cell epitopes; and detecting a change in state in T-cells in a portion removed from the sample.

In one aspect, the container comprises two or more SARS-CoV-2 antigens.

In another aspect, the one or more SARS-CoV-2 antigens is selected from the group consisting of nucleocapsid (N) protein, membrane (M) protein, envelope (E) protein, and spike (S) protein. In one aspect, the one or more antigens comprise M protein, E protein, and S protein. In still another aspect, the one or more antigens comprise N protein.

In still another aspect, each of the SARS-COV-2 antigens comprises between 8 and 50 amino acid residues

In another aspect, the sample is whole blood, cerebrospinal fluid, synovial fluid, or lymph fluid.

In still another aspect, detecting a change in state of T-cells in the sample comprises detecting the presence or level of a T-cell immune response indicator.

In one aspect, the immune response indicator may be IFN-γ, IL-1α, IL-1β, IL-2, IL-10, IL-12, IL-17, TNF-α, TNF-β, a granzyme, or a perforin. In one aspect, the T-cell immune response indicator is IFN-γ.

In yet another aspect, the presence or level of the T-cell immune response indicator is determined using an antibody that specifically binds a molecule selected from the group consisting of IFN-γ, IL-1α, IL-1β, IL-2, IL-10, IL-12, IL-17, TNF-α, TNF-β, a granzyme, and a perforin.

Another embodiment relates to a composition comprising one or more SARS-CoV-2 peptides, wherein the peptides consist of M, E, S, and/or N peptides; a container for the one o more SARS-CoV-2 peptides; T cells; and an antibody that binds to a T-cell immune response indicator; wherein each peptide comprises between 8 and 50 amino acids; and wherein the peptides comprise T-cell epitopes. In one aspect, the T-cell immune response indicator may be IFN-γ, IL-1α, IL-1β, IL-2, IL-10, IL-12, IL-17, TNF-α, TNF-β, a granzyme, or a perforin. In one aspect, the T-cell immune response indicator is IFN-γ.

In one aspect, the container is a channel or depression in microfluidic device, a well of a microtiter or microwell plate, a microtube, an EPPENDORF® Tube, a microcentrifuge tube, a capillary tube, a test tube, and a blood collection tube.

### BRIEF DESCRIPTION OF THE DRAWINGS

FIG. 1 shows an example of a general scheme for measuring T-cell cytokines and anti-SARS-CoV-2 antibodies.

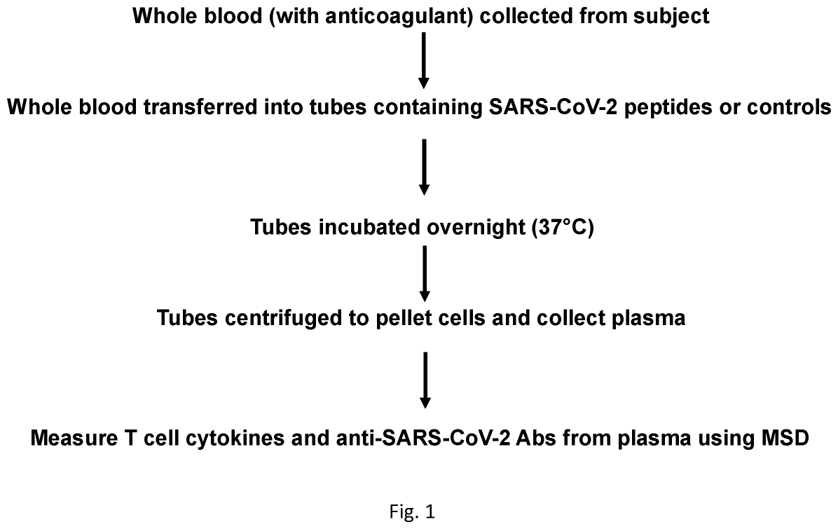

FIG. 2 shows the outline of a study using the QUANTIFERON® assay platform to detect the presence of anti-SARS-CoV-2 T-cells in individuals administered a SARS-CoV-2 vaccine.

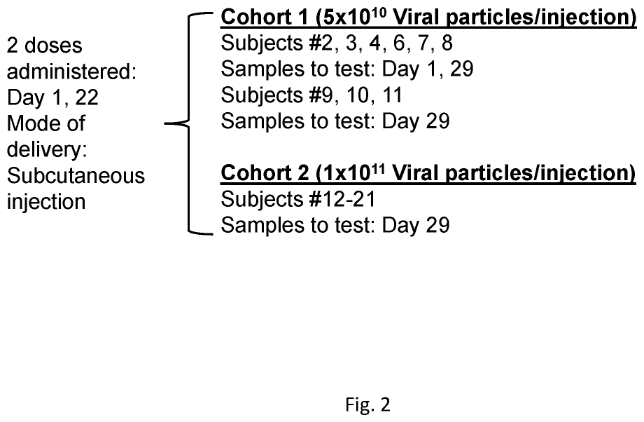

FIGS. 3A and 3B show SARS-Co-V-2 N protein-induced interferon-y production in samples from individuals administered a SARS-CoV-2 vaccine. Cohort 1 (FIG. 3A) received 5×10¹⁰ virus particles. Cohort 2 (FIG. 3B) received 1×10¹¹ virus particles.

FIGS. 4A and 4B show SARS-Co-V-2 S protein-induced interferon-y production in samples from individuals administered a SARS-CoV-2 vaccine. Cohort 1 (FIG. 4A) received 5×10¹⁰ virus particles. Cohort 2 (FIG. 4B) received 1×10¹¹ virus particles.

FIGS. 5A and 5B show CEFT-induced interferon-y production by T-cells from vaccinated individuals. CEFT consists of a set of peptides from Epstein-Barr virus, cytomegalovirus, and influenza virus.

### DETAILED DESCRIPTION

Methods, compositions, and devices have been developed that allow for rapid and easy detection of severe respiratory coronavirus 2 (SARS-CoV-2), and identification of individuals infected with SARS-CoV-2. The methods use SARS-CoV-2 antigens to identify anti-SARS-CoV-2 T-cells in a sample from an individual. The antigens are generally deposited in the assay container, beforehand, allowing for controlled and easy practice of the assay, in a manner that is reproducible and accurate. Accordingly, a method of the present disclosure can generally be practiced by contacting a T-cell containing sample with one or more SARS-CoV-2 antigens and detecting a change in the state of T-cells in the sample. Detection of a change of state of the T-cells indicates the presence of SARS-CoV-2.

As used herein, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. The claims may be drafted to exclude any optional element. As such, this statement is intended to serve as antecedent basis for use of such exclusive terminology as “solely,” “only” and the like regarding the recitation of claim elements or use of a “negative” limitation.

Certain features of the disclosure, which are described in the context of separate embodiments, may also be provided in combination in a single embodiment. Conversely, various features of the disclosure, which are, for brevity, described in the context of a single embodiment, may also be provided separately or in any suitable sub-combination. All combinations of the disclosed embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

The present disclosure is not limited to particular embodiments described herein. The terminology used herein is not intended to be limiting.

Publications discussed herein are provided solely for their disclosure prior to the filing date of the present application. Nothing herein is to be construed as an admission that the present disclosure is not entitled to antedate such publication by virtue of prior disclosure. Further, the publication dates provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by a person of skill in diagnostics. Although any methods and materials similar or equivalent to those described herein can also be used in the practice or testing of tools and assays of the disclosure, the preferred methods and materials are now described.

One embodiment is a method of detecting the presence of SARS-CoV-2 in an individual, comprising incubating a sample comprising T-cells from the individual, in a container comprising one or more SARS-CoV-2 antigens; and detecting a change in state of T-cells in the sample. Detection of a change in state of the T-cells indicates the presence of SARS-CoV-2 in the individual.

The terms individual, subject, and patient are herein used interchangeably to refer to any human or non-human animal susceptible to infection by SARS-CoV-2. Examples include, but are not limited to, humans, non-human primates, such as chimpanzees, apes and other monkey species; domestic mammals (e.g., dogs and cats); laboratory animals (e.g., mice, rats guinea pigs); birds, and, bats. Individuals of any age or race are covered by the present disclosure.

In one aspect, the individual is infected with SARS-CoV-2. As used herein, an infected individual is known to have SARS-CoV-2 in their body. In one aspect, the individual has been exposed to SARS-CoV-2. The terms exposed, and the like, indicate an individual has had contact with a source of SARS-CoV-2 (e.g., an infected individual). In one aspect, the individual has been vaccinated against SARS-CoV-2. A vaccinated individual has been administered a vaccine intended to protect against SARS-CoV-2.

As used herein, a sample comprising T-cells from the individual refers to a liquid comprising T-cells from the individual. Samples may optionally comprise antigen presenting cells (APCs), such as dendritic cells, macrophages, Langerhans cells, and B cells. The sample may be, for example, a biological fluid from the individual that comprises T-cells, and optionally APCs, examples of which include, but are not limited to, whole blood, cerebrospinal fluid, synovial fluid, or lymph fluid. Alternatively, the sample can be a liquid, (e.g., a buffered solution) to which has been added T-cells, and optionally APCs, the T-cells and optional APCs being obtained from fluid or tissue from the individual, or from culture. In one aspect, the sample is derived from, or is, whole blood, cerebrospinal fluid, synovial fluid, or lymph fluid. In one aspect, the sample is derived from, or is, whole blood.

As used herein, the term “T cell” refers to T lymphocytes as defined in the art and may be, for example, thymocytes, immature T lymphocytes, mature T lymphocytes, resting T lymphocytes, activated T lymphocytes, memory T cells, and regulatory T-cells, CD4+ T cells, CD8+T cells, or CD4+ CD8+T cells. The T cells may be Th1 cells, Th2 cells, Th9 cells, Th17 cells or Tfh cells.

As used herein, the term “container” is used as commonly understood by one of ordinary skill in diagnostics. Any container suitable for detecting a change in state of a T-cell may be used. The container can be of any volume that holds a sample suitable for detecting a T-cell change of state. For example, the container may have a volume suitable for holding a 1 μl, 5 μl, 10 μl 50 μl, 100 μl, 250 μl, 500 μl, 1 milliliter (ml), 3 ml, 5 m, or 10 ml sample. The container may be made from any material suitable for holding the sample. Examples of suitable materials include, but are not limited to, glass, plastic, polypropylene (PP), polydimethylsiloxane (PDMS), polymethyl methacrylate (PMMA), polystyrene (PS), cyclic olefin copolymer (COC), or a combination thereof. Examples of suitable types of containers include, but are not limited to, a channel or depression in a microfluidic device, a well of a microtiter or microwell plate, a microtube, an EPPENDORF® Tube, a microcentrifuge tube, including a screw cap microcentrifuge tube, a capillary tube, a test tube, and a blood collection tube (e.g., a VACUTAINER® tube).

As used herein, a SARS-CoV-2-antigen refers to a SARS-CoV-2 protein, a variant thereof, or any portion thereof that can be recognized by an anti-SARS-CoV-2 T-cell. Accordingly, SARS-CoV-2 antigens of the disclosure comprise at least one T-cell epitope. As used herein, the term “T cell epitope” refers to an amino acid sequence of 8 to 30 contiguous amino acid residues that is capable of specific binding with a specific T-cell receptor (TCR), usually, but not necessarily, in conjunction with a major histocompatibility complex (MEW).

Examples of suitable SARS-CoV-2 proteins that can be used as, or to produce, SARS-CoV-2 antigens include, but are not limited to, SARS-CoV-2 nucleocapsid protein (N protein), SARS-CoV-2 membrane protein (M protein), SARS-CoV-2 envelope protein (E protein), and SARS-CoV-2 spike protein (S protein). Examples of SARS-CoV-2 N, M, E and S proteins are represented by SEQ ID NOS: 1, 2, 3, and 4, respectively. In one aspect, the one or more SARS-CoV-2 antigen comprises the amino acid sequence of a protein selected from the group consisting of an N protein, an M protein, an E protein, and an S protein. In one aspect, the one or more SARS-CoV-2 antigen comprises a sequence selected from the group consisting of SEQ ID NO:1, SEQ ID NO:2, SEQ ID NO:3, and SEQ ID NO:4.

A SARS-CoV-2 antigen used in the disclosed methods and compositions may be a variant of a SARS-CoV-2 protein disclosed herein. The term variant refers to a protein, or fragment thereof, having an amino acids sequence that is similar, but not identical, to a referenced sequence (e.g., a SARS-CoV-2 protein sequence), wherein the activity of the variant protein is not significantly altered. These variations in sequence can be naturally occurring variations or they can be engineered through the use of technique known to those skilled in the art. Examples of suitable variations include, but are not limited to, amino acid deletions, insertions, substitutions, and combinations thereof.

Amino acids can be classified into groups based on their physical properties. Examples of such groups include, but are not limited to, charged amino acids, uncharged amino acids, polar uncharged amino acids, and hydrophobic amino acids. Preferred variants are those in which an amino acid is substituted with an amino acid from the same group. Such substitutions are referred to as conservative substitutions.

Naturally occurring residues may be divided into classes based on common side chain properties:

1) hydrophobic: Met, Ala, Val, Leu, Ile;

2) neutral hydrophilic: Cys, Ser, Thr;

3) acidic: Asp, Glu;

4) basic: Asn, Gln, His, Lys, Arg;

5) residues that influence chain orientation: Gly, Pro; and

6) aromatic: Trp, Tyr, Phe.

Non-conservative substitutions may involve the exchange of a member of one of these classes for a member from another class.

As used herein, the phrase “significantly affect a proteins activity” means to decrease in the activity of a protein by 20% more. Regarding the present disclosure, such an activity may be measured, for example, as the affinity of an anti-SARS-CoV-2 T-cell to recognize a SARS-CoV-2 protein or variant thereof.

In one aspect, each of the one or more SARS-CoV-2 antigens comprises, or consists of, a variant of a protein selected from the group consisting of an N protein, an M protein, an E protein, and an S protein. In one aspect, each of the one or more SARS-CoV-2 antigen comprises, or consists of, a sequence at least 85% identical, at least 90% identical, at least 95% identical, at least 97% identical or at least 100% identical to a sequence selected from the group consisting of SEQ ID NO:1, SEQ ID NO:2, SEQ ID NO:3, and SEQ ID NO:4.

A SARS-CoV-2 antigen used in the disclosed methods and compositions may be a portion (e.g., peptide) of a SARS-CoV-2 protein, such as those disclosed herein, as long as the portion comprises a sufficient number of amino acid residues to be recognized by a T-cell receptor. In one aspect, each of the one or more SARS-CoV-2 antigens comprises, or consists of, at least a portion of a protein selected from the group consisting of an N protein, an M protein, an E protein, and an S protein, wherein the at least a portion comprises a T-cell epitope. In one aspect, each of the one or more SARS-CoV-2 antigens comprises, or consists of, at least a portion of a protein comprising a sequence at least 80% identical, at least 90% identical, at least 95% identical, at least 97% identical or at least 100% identical to a sequence selected from the group consisting of SEQ ID NO:1, SEQ ID NO:2, SEQ ID NO:3, and SEQ ID NO:4, wherein the at least a portion comprises a T-cell epitope.

In one aspect, each of the one or more SARS-CoV-2 antigens comprises, or consists of, between 8 and 50, optionally 8 and 30, contiguous amino acid residues from a sequence at least 85% identical, at least 90% identical, at least 95% identical, at least 97% identical or at least 100% identical to a sequence selected from the group consisting of SEQ ID NO:1, SEQ ID NO:2, SEQ ID NO:3, and SEQ ID NO:4, wherein the contiguous amino acid residues comprise a T-cell epitope. In one aspect, each of the one or more SARS-CoV-2 antigens comprises at least, or consists of, 8, 9, 10, 11, 12, 13, 14, 15, 16, 17, 18, 19, 20, 21, 22, 23, 24, 25, 26, 27, 28, 29, or 30, contiguous amino acid residues from a protein selected from the group consisting of an N protein, an M protein, an E protein, and an S protein, wherein the contiguous amino acid residues comprise a T-cell epitope. In one aspect, each of the one or more SARS-CoV-2 antigens comprise at least, or consist of, 8, 9, 10, 11, 12, 13, 14, 15, 16, 17, 18, 19, 20, 21, 22, 23, 24, 25, 26, 27, 28, 29, or 30, contiguous amino acid residues from a sequence at least 85% identical, at least 90% identical, at least 95% identical, at least 97% identical or at least 100% identical to a sequence selected from the group consisting of SEQ ID NO:1, SEQ ID NO:2, SEQ ID NO:3, and SEQ ID NO:4, wherein the contiguous amino acid residues comprise a T-cell epitope.

In one aspect, the peptides are obtained from a commercial source such as Pepmix (JPT Peptide Technologies, GmbH), wherein the Pepmix products comprise overlapping peptide pools covering putative T-cell binding antigenic sequences from viral antigens. Pepmix peptide pools are routinely used for antigen-specific T-cell stimulation in T-cell assays, in vitro T-cell expansion, immune monitoring, and cellular immune response profiling.

In one aspect, the one or more SARS-CoV-2 antigens are introduced into the container prior to addition of the sample to the container. In one aspect, the container may comprise two or more SARS-CoV-2 antigens, or three or more SARS-CoV-2 antigens The one or more antigens may be introduced immediately before addition of the sample, or at an earlier time, such as, 1 minute, one hour, one day, one week, or one month prior to addition of the sample. The one or more antigens may be introduced with a stabilizing agent, such as a buffer, to allow for storage of the container. The one or more antigens may be attached and/or deposited to the interior surface of the container through non-covalent (e.g., ionic bonds, hydrogen bonds, hydrophobic bonds), or covalent bonds. The interior surface may be coated with a scaffold molecule, such as alkoxysilane, fibrin or collagen, to aid attachment of the one or more antigens.

Incubation of the container in the disclosed methods, is performed under conditions sufficient to allow recognition of a T-cell epitope by a T-cell. Such conditions may include incubating the container in a temperature range of between 5° C. and 90° C., for a time period between one minute and 24 hours. Generally, the container is incubated at a temperature between 30° C. and 40° C., and preferably at about 37° C., for about 1 minute, about 2 minutes, about 5 minutes, about 10 minutes, about 20 minutes, about 30 minutes, one hour, two hours, four hours, six hours, twelve hours, sixteen hours, or 24 hours. Suitable incubation conditions are known to those skilled in the art, and are also disclosed in U.S. Patent Publication No. 2020/0191771.

The disclosed methods comprise determining if T-cells in the sample recognize the one or more antigens present in the container by detecting a change in state of the T-cells in the presence of the one or more SARS-CoV-2 antigens. A change in state indicates T-cell activation resulting from antigen specific activity of the T-cell after the T-cell receptor binds an antigen. The molecular events (e.g., protein kinase activation) following activation of a T-cell include are well known to those skilled in the art and any such events may be used to detect a change in state of a T-cell. Such events can include, for example, activation of protein tyrosine kinases, phosphorylation of immunoreceptor tyrosine-based activation motifs (ITAMs), and influx of calcium into the cell. The change in state may also be the start of, or increase in, secretion of a substance from the T-cell. Such a substance may be referred to as an immune response indicator (“indicator”). The indicator may be any molecule that is secreted by a T-cell upon recognition of an antigen. Examples of indicators include, but are not limited to, IFN-γ, IL-1α, IL-1β, IL-2, IL-10, IL-12, IL-17, TNF-α, TNF-β, a granzyme, and a perforin. Thus in one aspect, a change in state of a T-cell is detected by determining the presence or level of an indicator selected from the group consisting of IFN-γ, IL-1α, IL-1β, IL-2, IL-10, IL-12, IL-17, TNF-α, TNF-β, a granzyme, and a perforin, in the sample. In one aspect, the change in state of a T-cell is detected by determining the presence or level of IFN-γ in the sample.

The method used to detect a change of state of a T-cell depends on the molecule being detected. For example, activation of a protein may be accomplished using an assay for the activity of the activated protein. A secreted indicator, such as IFN-γ, may be detected by allowing it to bind a first binding agent that is specific for the indicator, and then measuring the presence of indicator/first binding agent complex. In one aspect, detecting a change in the state of T-cells in the sample comprises contacting at least a portion of the incubated sample with a first binding agent specific for the indicator. In one aspect, the first binding agent is an antibody that specifically binds the indicator (e.g., an anti-IFN-γ antibody). In one aspect, the first binding agent is a receptor molecule that specifically binds the indicator (e.g., an IFN-γ receptor).

The specific binding agent may be immobilized on a solid support, such as a bead or a plate. Once the indicator has bound to the first binding agent, the solid support can be washed to remove non-specifically bound material. The indicator/first binding agent complex can then be detected using a second binding agent that recognizes the indicator/first binding agent complex and that may or may not be labeled with a detectable label. In one aspect, the second binding agent is an antibody that recognizes the indicator/first binding agent complex.

There are two major types of T-cells: helper T-cells (also called CD4+ cells), which express the CD4 co-receptor; and cytotoxic T-cells (also called CD8+ cells), which express the CD8 co-receptor. CD4+ T-cells recognize antigen presented by the class I major histocompatibility complex (MEW I) on an APC, while CD8+ cells recognize antigen presented by the class II major histocompatibility complex (MEW II) on an APC. Specific inhibition of the MEW I or the MEW II therefore provides a method to identify the type of T-cell involved in an anti-SARS-CoV-2 response. Thus, in one aspect of the disclosed methods, the container comprising the SARS-CoV-2 antigen comprises antibodies that neutralize either MHCI or MHCII. As used herein, “neutralization” of MHCI or MHC II means that antibodies bind to the MHC complex and prevent presentation of antigen to the T-cell receptor. Thus, if T-cell activation is detected in the absence of anti-MHCI antibody but is not detected in the presence of anti-MHCI antibody, one can conclude that the anti-SARS-CoV-2 response is mediated by CD4+ T-cells. Likewise, if T-cell activation is detected in the absence of anti-MHCII antibody but is not detected in the presence of anti-MHCII antibody, one can conclude that the anti-SARS-CoV-2 response is mediated by CD8+ T-cells.

Because methods of the disclosure detect the presence of SARS-CoV-2, they are useful for identifying individuals that have been infected with SARS-CoV-2, making treatment decisions, and monitoring vaccination effectiveness. One embodiment is method of identifying an individual that has been infected with SARS-CoV-2, comprising detecting the presence of SARS-CoV-2 using methods of the disclosure. In one aspect, such a method comprises incubating a sample comprising T-cells from the individual, in a container comprising one or more SARS-CoV-2 antigens; and detecting a change in state of a T-cell in the sample. Detecting a change in state of the T-cells indicates the individual has be infected with SARS-CoV-2. In one aspect, the individual has been exposed to, or vaccinated against, SARS-CoV-2.

One embodiment is method of treating an individual for infection with SARS-CoV-2, comprising detecting the presence of SARS-CoV-2 using methods of the disclosure. In one aspect, such a method comprises incubating a sample comprising T-cells from the individual, in a container comprising one or more SARS-CoV-2 antigens; and detecting a change in state of a T-cell in the sample, wherein if a change in state is detected, treating the individual for a SARS-CoV-2 infection. Treating the individual for a SARS-CoV-2 infection may comprise quarantining the individual, admitting the individual to a medical facility, and/or administering any suitable compound suitable for treatment of a SARS-CoV-2 infection.

One embodiment is a method of determining the efficacy of a therapeutic response on SARS-CoV-2 infection. In one aspect, the disclosed methods may be used to determine the efficacy of the therapeutic method on preventing infection. In such aspect, the SARS-CoV-2 infection status of two cohorts is monitored over a period of time. In this aspect, a first cohort of one or more individuals known to not have been infected with SARS-CoV-2 (i.e., an uninfected individual) is given a therapeutic treatment and their infection status monitored over a period of time using the disclosed methods. The infection status of the second cohort, also comprising one or more uninfected individuals, is also monitored for the period of time using the disclosed methods. By comparing any difference in the infection rate of the two cohorts, the efficacy of the therapeutic response is determined. In one aspect, the disclosed methods may be used to determine the efficacy of a therapeutic treatment on an individual infected with SARS-CoV-2. In such aspect, the infection status of an individual known to be infected with SARS-CoV-2 may be determined using the disclosed methods. A therapeutic treatment is then administered to the individual, and their infection status monitored over time using the disclosed methods. A decrease in the level of SARS-CoV-2 reactive T-cell indicates the therapeutic treatment reduced the level of SARS-CoV-2 in the individual. In this way, the efficacy of the therapeutic treatment may be monitored. In certain aspects, the infection status of the individual may be monitored for any desired period of time following administration of the therapeutic treatment. In certain aspects, the individual may be monitored for at least one week, at least two weeks, at least three weeks, at least one month, at least or at least two months, following administration of the therapeutic treatment.

One embodiment is a method of determining efficacy of a SARS-CoV-2 vaccine using methods of the disclosure. In one aspect, such method comprises assaying a pre-vaccination, T-cell containing sample from an individual for a change in state of T-cells in the sample; assaying a post-vaccination, T-cell containing sample from the individual for a change in state of T-cells in the sample; and comparing the presence or level of any change of state in the pre- and post-vaccination samples; thereby determining efficacy of the SARS-CoV-2 vaccine; wherein the step of assaying comprises incubating the T-cell containing sample in a container comprising one or more SARS-CoV-2 antigens; and detecting a change in state of T-cells in each sample. In certain aspects, the individual may be monitored for any desired period of time following administration of the vaccine. In certain aspects, the individual may be monitored for at least one week, at least two weeks, at least three weeks, at least one month, at least or at least two months, following administration of the vaccine.

In these methods, the sample may be a biological fluid from the individual that comprises T-cells, and optionally APCs, examples of which include whole blood, cerebrospinal fluid, synovial fluid, or lymph fluid. In one aspect, the sample is derived from, or is, whole blood, cerebrospinal fluid, synovial fluid, or lymph fluid. In one aspect, the sample is derived from, or is, whole blood. In one aspect, the sample may be a liquid, (e.g., a buffered solution) to which has been added T-cells, and optionally APCs, obtained from fluid or tissue from the individual, or from a culture.

In these methods, the T-cell may be a thymocyte, an immature T lymphocyte, a mature T lymphocyte, a resting T lymphocyte, an activated T lymphocyte, a memory T cell, a regulatory T-cell, a CD4+ T cell, a CD8+ T cell, a CD4+ CD8+ T cell, a Th1 cell, a Th2 cell, Th9 cell, a Th17 cell or a Tfh cell.

In these methods, the container may comprise glass, polypropylene (PP), polydimethylsiloxane (PDMS), polymethyl methacrylate (PMMA), polystyrene (PS), cyclic olefin copolymer (COC), or a combination thereof, and may be a channel or depression in a microfluidic device, a well of a microtiter or microwell plate, a microtube, an EPPENDORF®, a microcentrifuge tube, including a screw cap microcentrifuge tube, a capillary tube, a test tube, or a blood collection tube (e.g., a VACUTAINER® tube).

In these methods, the one or more antigens may comprise, or consist of, a SARS-CoV-2 M, E or S protein, and optionally an N protein, variants therefore, and portions thereof, the portions comprising, or consisting of between 8 and 50 contiguous amino acid residues. The one or more antigens may comprise, or consist of, an amino acid sequence at least 85%, at least 90%, at least 95% , at least 97%, or 100% identical to SEQ ID NO:1, SEQ ID NO:2, SEQ ID NO:3, or SEQ ID NO:4, variants therefore, and portions thereof, the portions comprising, or consisting of between 8 and 50 contiguous amino acid residues.

In these methods, detecting a change in state of the T-cells may comprise detecting the start of, or increase in, secretion of an immune response indicator (‘indicator”). The indicator may be IFN-γ, IL-1α, IL-1β, IL-2, IL-10, IL-12, IL-17, TNF-α, TNF-β, a granzyme, or a perforin. The indicator may be detected by allowing it to bind to a first binding agent that specifically binds the indicator and measuring the presence of indicator/first binding agent complex. The first binding agent may be an antibody that specifically binds the indicator, or a receptor molecule that specifically binds the indicator. The first binding agent may be immobilized on a solid support, such as a bead or a plate. The indicator/first binding agent complex may be detected using a second, optionally labeled, binding agent. The second binding agent may be an antibody that recognizes the indicator/first binding agent complex.

One embodiment is a composition comprising at least one isolated SARS-CoV-2 M peptide, at least one isolated SARS-CoV-2 E peptide, at least one isolated SARS-CoV-2 S peptide, and human T-cells; wherein each peptide comprises, or consists of, between 8 and 50 amino acid residues, optionally between 8 and 30 amino acid residues, and wherein each peptide comprises a T-cell epitope. As used herein, an isolated peptide is a peptide that has been removed from its natural milieu. An isolated peptide can, for example, be obtained from its natural source, be produced using recombinant DNA technology, or be synthesized chemically. In one aspect, a composition of the disclosure may comprise one or more additional components, such as, a SARS-CoV-2 N peptide, a T-cell immune response indicator, and/or a binding agent that binds the immune response indicator. In one aspect, the immune response indicator is selected from the group consisting of IFN-γ, IL-1α, IL-10, IL-2, IL-10, IL-12, IL-17, TNF-α, TNF-β, a granzyme, and a perforin, in the sample. In one aspect, the immune response indicator is IFN-γ. In one aspect, the binding agent is an antibody (e.g., an anti-IFN-γ antibody) or a receptor molecule, such as an IFN-γ receptor.

One embodiment is a method of producing a SARS-CoV-2 diagnostic device, comprising depositing one or more peptides selected from the group consisting of an isolated SARS-CoV-2 M peptide, an isolated SARS-CoV-2 E peptide, and an isolated SARS-CoV-2 S peptide, on the interior surface of a container, wherein each peptide comprises, or consists of, between 8 and 50 amino acid residues, optionally between 8 and 30 amino acid residues, and wherein each peptide comprise a T-cell epitope. In one aspect, the method comprises further comprises depositing a SARS-CoV-2 N peptide on the interior surface of the container, the peptide comprising, or consisting of, between 8 and 50, optionally 8 and 30, amino acid residues. One embodiment is a diagnostic device produced using the disclosed method.

As used herein, depositing the one or more peptides on the interior surface of the container means contacting the one or more peptides with the interior surface such that the peptides attach to the interior surface. Such attachment may be non-covalent and result from, for example, ionic bonds, hydrogen bonds, or hydrophobic bonds. Alternatively, attachment may be due to covalent bonds between the one or more peptides and the interior surface. In one aspect, the interior surface is coated with a scaffold molecule, such as alkoxysilane, fibrin or collagen.

The one or more antigens may be introduced with a stabilizing agent, such as a buffer, to allow for storage of the container. The container comprising the one or more antigens may be subjected to conditions that reduce any moisture present associated with one or more antigens. The container comprising the one or more antigens may be lyophilized.

The container may comprise glass, polypropylene (PP), polydimethylsiloxane (PDMS), polymethyl methacrylate (PMMA), polystyrene (PS), cyclic olefin copolymer (COC), or a combination thereof, and may be a channel or depression in a microfluidic device, a well of a microtiter or microwell plate, a microtube, an EPPENDORF® Tube, a microcentrifuge tube, including a screw cap microcentrifuge tube, a capillary tube, a test tube, or a blood collection tube (e.g., a VACUTAINER® tube).

Suitable peptide lengths have been disclosed elsewhere herein. For example, the one or more peptides may comprise, or consist of, between 8 and 50, optionally 8 and 30, contiguous amino acid residues from a SARS-CoV-2 M, E or S protein. The one or more peptides may further comprise a peptide comprising, or consisting of, between 8 and 50, optionally 8 and 30, contiguous amino acid residues from a SARS-CoV-2 N protein, variants therefore, and portions thereof. The one or more peptides may comprise, or consist of, between 8 and 50, optionally 8 and 30, contiguous amino acid residues from an amino acid sequence at least 85%, at least 90%, at least 95% , at least 97%, or 100% identical to SEQ ID NO:1, SEQ ID NO:2, SEQ ID NO:3, or SEQ ID NO:4.

One embodiment is a kit. Kits may include, for example, a composition or a diagnostic device of the disclosure. Kits may also include associated components including, but not limited to, nucleic acid molecules for producing peptides of the disclosure, buffers, labels, containers, vials, syringes, and instructions for using the kit.

The following examples are put forth to provide those of ordinary skill in the art with a complete disclosure and description of how to make and use the embodiments and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations are used.

### EXAMPLES

### Example 1. Rapid T-Cell Assay for Measuring Antigen-Specific T Cell Responses from Whole Blood Using ELISA

Peptide stimulation of whole blood: Whole blood (500 μL) is placed into a 2 mL polypropylene tube. Peptide mixes are removed (JPT Pepmix; Spike: #PM-WCPV-S-1; Nucleocapsid: #PM-WCPV-NCAP-1; CEFT control peptides: #PM-CEFT-4; stocks of individual peptides are typically 1 mM in DMSO and stocks of peptide pools are typically 1 mg/ml/peptide in DMSO) from −20° C. freezer, thawed at room temperature (RT); and tubes spun down briefly. A sufficient volume of peptide is added to achieve a concentration of 2 μg/mL, i.e. 1 μg of peptide per 2 mL polypropylene vial of whole blood. An equivalent volume of DMSO is added as a negative control. 1.25 μL IMMUNOCULT™ T cell activator is added as a positive control. Stimulation conditions are as follows: 1) negative control (costimulatory reagent plus DMSO); 2) positive control (IMMUNOCULT™ T cell activator); 3) Nucleocapsid pepmix; 4) Spike pepmix (S1 and S2 combined from separate vials); 5) CEFT pepmix. There are a total of five vials (one vial per experimental condition) if sufficient blood is available, to be included provided that more than 27 mls of blood are collected. Each stimulation tube is distributed in duplicate to ELISA plates. 5 μL costimulatory reagent (anti-CD28/anti-CD49d; BD BIOSCIENCE®; #347690) is added to each vial of whole blood being stimulated, with the exception of the positive control. The whole blood is incubated at 37° C. overnight (16-20 hours) in a water bath.

Coat ELISA plate. Wells are coated with capture Ab (4 μg/mL, final concentration) diluted in coating buffer (add 50 μL/well). Plates are left overnight at 4° C. in refrigerator.

Collect stimulated cell supernatant. The experiment vials (five) are removed from the 37° C. water bath. Vials are centrifuged at 500×g for ten minutes to pellet cells. Supernatant is collected and transferred to a clean microcentrifuge tube. Since red blood cells do not pellet well, some red blood cells may be transferred to the second tube. Therefore, the collection tube to which the supernatant was transferred, is centrifuged 500×g, ten minutes. The supernatant is collected into a clean microcentrifuge tube for assay.

Run ELISA. Block plate: Shake out the coating solution from plate, blot on paper towel; and add 200 μL of blocking buffer (PBS, 1% BSA) to each well. Incubate for 1 hour at RT with shaking. The blocking solution is decanted. The plate is washed 3× with 150 μL wash buffer. Standards and test articles are added. Incubate 2-3 hours with shaking at RT. Standards are diluted in blocking buffer, beginning with the highest concentration of standard (5120 pg/ml) and making serial two-fold dilutions to the lowest concentration of standard (20 pg/ml). Six wells of blanks (blocking buffer), two wells of each standard (100 μl each), and two wells of each test article (50 μl each are run; 50 μl of blocking buffer is added to reach a total volume of 100 μl per well). Plates are washed with wash buffer (4×, 150 μL). Biotinylated detecting Ab (1 ug/mL, diluted in blocking buffer, 100 uL/well) is added. The plates are incubated for one hour with shaking at RT. The plates are washed with wash buffer (3×). Streptavidin-peroxidase (100 μL/well at 1:1000 dilution in blocking buffer) is added and incubated for 60 min, RT with shaking. Streptavidin-peroxidase solution is discarded and the plates are washed with wash buffer (3×), followed by PBS (3×).

Develop: Prepare 1:1 mix of 2,2Lazino-bis(3-ethylbenzothiazoline-6-sulphonic acid peroxidase substrate and Peroxidase B solution. Add the mixture to the plates and incubate 10 minutes (or until color of third to the lowest concentration standard is discernable), RT with shaking. Stop the reaction by adding stop solution (1% SDS; 50 Read plate at 405 nm.

Raw ELISA data is imported from the plate reader (BIOTEK® Synergy 2) into Microsoft Excel, which is used to generate data plots and statistics. The standard curve is created by graphing the concentration of standards against the optical density values measured by the reader for the standard values. A linear regression curve is created using the standards and optical density values. The equation for the curve allows one to calculate the values of the test articles based on the optical density value of the test article.

System Suitability Criteria. The lower limit of detection must be no less than 80 pg/ml, preferably at 20 pg/ml. Standard curve-test articles must fall within the standard curve for reliable/accurate values to be calculated.

Sample Acceptance Criteria. Negative control-levels of interferon-gamma (IFN-g) measured should be less than or within two standard deviations above the lower limit of detection; greater than two standard deviations could indicate that the subject had an acute inflammatory response, making data analysis difficult. Specimens that produce negative control levels that are greater than two standard deviations above the lower limit of detection are considered a failed sample and excluded from analysis. Positive control-levels of IFN-g measured should be greater than 300 pg/ml for the values to be considered reliable. Specimens that produce positive control levels less than 300 pg/ml will be considered a failed sample and excluded from analysis. Samples stimulated with Nucleocapsid pepmix, Spike pepmix (S1 and S2 combined from separate vials), or CEFT (CMV, Epstein-Barr, Flu, Tetanus) pepmix (JPT Peptide Technologies) will be accepted for analysis if optical density readings are at least two standard deviations above the lower limit of detection.

### Example 2. Use of QUANTIKINE® Assay for SARS-CoV-2 Vaccine Immunogenicity

Rapid T-cell assay performed as in example 1, except QUANTIKINE® (R&D Systems) ELISA is used to detect γ-IFN.

### Example 3. Use of QUANTIFERON® Assay for SARS-CoV-2 Vaccine Immunogenicity

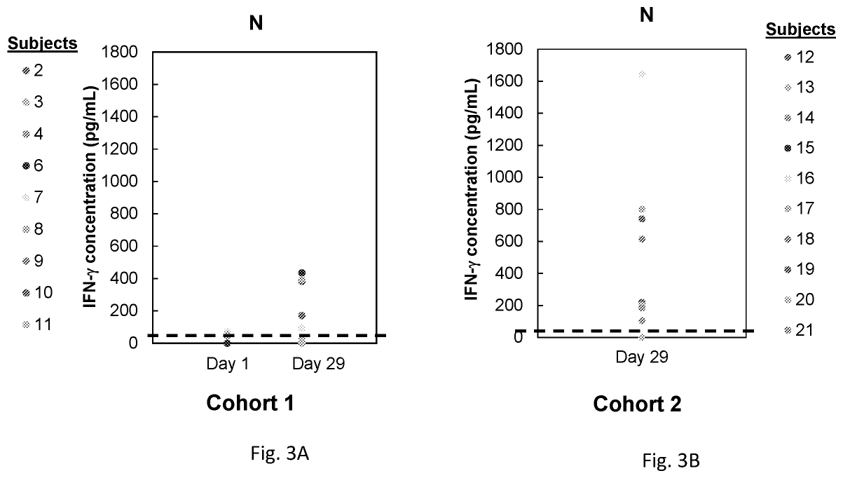

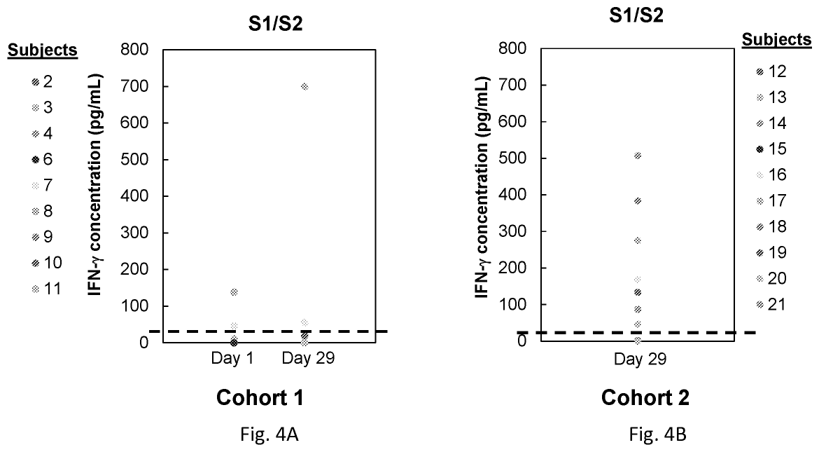

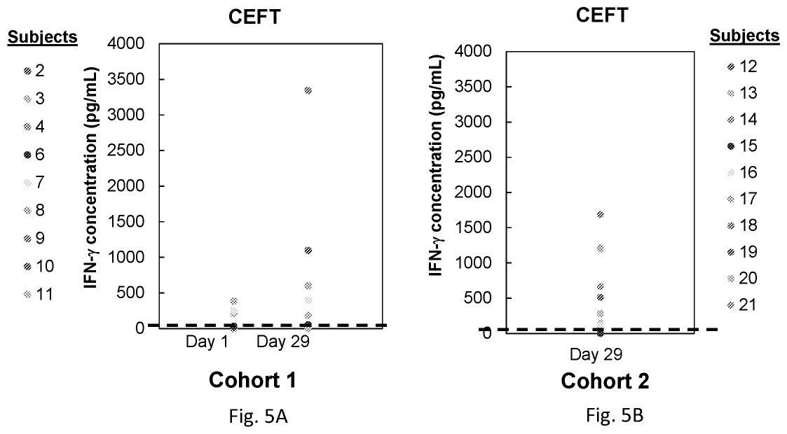

This example demonstrates the use of a QUANTIFERON® assay (QIAGEN®) to measure a human immune response to a SARS-CoV-2 vaccine. Individuals were administered either 5×10¹⁰ virus particles (Cohort 1) or 1×10¹¹ virus particles (Cohort 2). The viruses were administered by subcutaneous injections. A second dose was given 22 days following the first dose. At various times after infection, blood samples were obtained the presence of T-cells that recognize SARS-CoV-2 N protein, or SARS-CoV-2 S protein, was determined using a QUANTIFERON® assay. The results of this study are shown in FIGS. 3A-3B, 4A-4B and 5A and 5B. The results demonstrate that an increase in T-cell activation is observed with an increasing dose of vaccine.

What is claimed:

1. A method of detecting severe acute respiratory syndrome coronavirus 2 (SARS-CoV-2) in an individual, the method comprising:
a. incubating a sample in a container under conditions sufficient for specific recognition by T-cells, wherein the sample comprises T-cells from the individual, wherein the container comprises one or more SARS-CoV-2 antigens, wherein the one or more SARS-CoV-2 antigens are deposited on the interior surface of the container, and wherein the SARS-CoV-2 antigens comprise T-cell epitopes; and
b. detecting a change of state in T-cells in a portion removed from the sample.

2. The method of claim 1, wherein the container comprises two or more SARS-CoV-2 antigens.

3. The method of claim 1, wherein the one or more SARS-CoV-2 antigens is selected from the group consisting of nucleocapsid (N) protein, membrane (M) protein, envelope (E) protein, and spike (S) protein.

4. The method of claim 3, wherein the one or more antigens comprise M protein, E protein, and S protein.

5. The method of claim 3, wherein the one or more antigens comprise N protein.

6. The method of claim 1, wherein each SARS-CoV-2 antigens comprises between 8 and 50 amino acid residues.

7. The method of claim 1, wherein the sample is whole blood, cerebrospinal fluid, synovial fluid, or lymph fluid.

8. The method of claim 1, wherein detecting a change in state of T-cells in the sample comprises detecting the presence or level of a T-cell immune response indicator.

9. The method of claim 8, wherein the T-cell immune response indicator is selected from the group consisting of IFN-γ, IL-1α, IL-10, IL-2, IL-10, IL-12, IL-17, TNF-α, TNF-β, a granzyme, and a perforin.

10. The method of claim 9, wherein the T-cell immune response indicator is IFN-γ.

11. The method of claim 8, wherein the presence or level of the T-cell immune response indicator is determined using an antibody that specifically binds a molecule selected from the group consisting of IFN-γ, IL-1α, IL-10, IL-2, IL-10, IL-12, IL-17, TNF-α, TNF-β, a granzyme, and a perforin.

12. A composition comprising:
a. one or more SARS-CoV-2 peptides, wherein the peptides consist of M, E, S, and/or N peptides;
b. a container for the one or more SARS-COV-2 peptides;
c. T-cells; and
d. an antibody that binds to a T-cell immune response indicator;
wherein each peptide comprises between 8 and 50 amino acids; and
wherein the peptides comprise T-cell epitopes.

13. The composition of claim 12, wherein the T-cell immune response indicator is selected from the group consisting of IFN-γ, IL-1α, IL-10, IL-2, IL-10, IL-12, IL-17, TNF-α, TNF-β, a granzyme, and a perforin.

14. The composition of claim 13, wherein the T-cell immune response indicator is IFN-γ.

15. The composition of claim 12, wherein the container is a channel or depression in a microfluidic device, a well of a microtiter or microwell plate, a microtube, an EPPENDORF® Tube, a microcentrifuge tube, a capillary tube, a test tube, and a blood collection tube.
